(19) United States
(12) Patent Application Publication
(10) Pub. No.: US 2023/0323348 A1
(43) Pub. Date: Oct. 12, 2023

(54) COMPOSITIONS AND METHOD FOR TREATING THALASSEMIA

(71) Applicant: St. Jude Children's Research Hospital, Inc., Memphis, TN (US)

(72) Inventors: Mitchell J. WEISS, Memphis, TN (US); Christophe LECHAUVE, Memphis, TN (US)

(21) Appl. No.: 18/042,015

(22) PCT Filed: Aug. 19, 2021
(86) PCT No.: PCT/US2021/046649

§ 371 Date: Feb. 17, 2023

Related U.S. Application Data
(60) Provisional application No. 63/068,015, filed on Aug. 20, 2020.

(51) Int. Cl. C12N 15/113; A61P 7/06

(52) U.S. Cl. CPC C12N 15/113; C12N 2310/113; A61P 7/06

(57) ABSTRACT

Methods for removing excess free α-globin in erythroid cells and treating a thalassemia using a miR-144 and/or miR-451 antagomir are described.

### INTRODUCTION

This application claims benefit of priority to U.S. Provisional Patent Application Ser. No. 63/068,015, filed Aug. 20, 2020, the content of which is incorporated herein by reference in its entirety.

This invention was made with government support under Grant Numbers DK092318 and DK061692 awarded by the National Institutes of Health. The government has certain rights in the invention.

### BACKGROUND

The production of functional hemoglobin A (HbA) tetramers (α₂β₂) requires the coordinated synthesis and assembly of α- and β-globin protein chains and iron-containing heme groups. Individually, all HbA components are toxic to red blood cells. In particular, β-thalassemias are common hemoglobinopathies in which β-globin gene (HBB) mutations cause the buildup of free α-globin. These unpaired α-globin chains initiate an oxidative damage cascade and form damaging precipitates that contribute largely to the clinical problems associated with β-thalassemia.

The pathophysiology of β-thalassemia bears similarities to a diverse group of protein-aggregation diseases affecting multiple organs. These disorders, which include Parkinson disease, Alzheimer disease, Huntington disease, amyotrophic lateral sclerosis, and α₁-antitrypsin deficiency, are caused by the accumulation of unstable, relatively insoluble proteins that become toxic for the cells. It is believed that the affected cells can detoxify and remove these damaging proteins via multiple interacting biochemical pathways called protein quality-control (PQC) pathways, but that disease ensues when such compensatory mechanisms are overwhelmed. Cellular PQC systems include molecular chaperones, ubiquitin-mediated proteolysis, and autophagy. Several lines of evidence suggest that β-thalassemic erythroid cells use PQC pathways to detoxify free a-globin. Specifically, the clinical severity of β-thalassemia is proportional to the degree of α-globin excess; there is a threshold below which excess α-globin is less harmful, as illustrated by subjects with the β-thalassemia trait, who experience 50% reduced α-globin synthesis with minimal clinical manifestations or accumulation of α-globin precipitates; and there is direct biochemical evidence that α-globin interacts with and is eliminated by cellular PQC components.

Studies have shown that normal and β-thalassemic erythroid precursors can balance globin ratios through selective α-chain proteolysis. Pulse-chase experiments using intact human β-thalassemic erythroid cells and cell lysates showed that excessive α chains are actively degraded and accumulate mainly in the late stages of erythroid maturation, presumably as the proteolytic capacity becomes exceeded. The ubiquitin proteasome system (UPS) is responsible for physiologic degradation of native proteins and for removing misfolded proteins as part of the PQC pathway in all cells. Studies have shown that normal and β-thalassemic hemolysates can ubiquitinate and degrade exogenous α-globin although the associated pathways remain largely uncharacterized. Red blood cell (RBC) precursors also use autophagy, a group of related processes in which targeted proteins or organelles are delivered to lysosomes and degraded. For example, autophagy-related genes are up-regulated by the master erythroid transcription factor GATA-1 during terminal erythropoiesis. During reticulocyte maturation, mitochondria are eliminated by “macroautophagy” or “mitophagy,” a process in which cells form double-membrane vesicles (autophagosomes) around cytoplasmic contents for delivery to lysosomes. Notably, electron micrographs of β-thalassemic erythroblasts identify a subset of α-globin precipitates within lysosomes. More recent work indicates that autophagic processes are increased in HbE/β-thalassemia. In particular, it has been shown that interregulated PQC pathways, including the ubiquitin proteasome system (UPS), autophagy, and heat-shock protein responses, are used to detoxify and remove free α-globin in β-thalassemic erythroid cells and that the UPS is regulated dynamically at the transcriptional level in β-thalassemic erythroblasts through a Nrf1 stress-response pathway (Khandros, et al. (2012) Blood 119(22):5265-5275).

Autophagy is promoted by AMP-activated protein Kinase (AMPK), which is a key energy sensor and regulates cellular metabolism to maintain energy homeostasis. Conversely, autophagy is inhibited by the Mechanistic Target Of Rapamycin (mTOR), a central cell-growth regulator that integrates growth factor and nutrient signals. In vivo inhibition of mTOR remarkably improves erythroid cell maturation and anemia in a model of β-thalassemia (Zhang, et al. (2014) Am. J. Hematol. 89(10):954-963). Specifically, inhibition of mTOR accelerates the autophagy of free α-globin by Unc-51 Like autophagy activating Kinase 1 (ULK1) (Lechauve, et al. (2019) Sci. Transl. Med. 11:eaav4881).

Additionally, ULK1 has been suggested to mediate AMPK and mTORC1 regulation of autophagy. Under glucose starvation, AMPK promotes autophagy by directly activating ULK1 through phosphorylation of Ser317, Ser555 and Ser777. Under nutrient sufficiency, high mTOR activity prevents ULK1 activation by phosphorylating ULK1 Ser757 and disrupting the interaction between ULK1 and AMPK. See Kim, et al. (2011) Nature Cell Biol. 13:132-141. Using knockout mouse models, it has been further shown that ULK1 is a component of the autophagy machinery that leads to the elimination of organelles in erythroid cells via a non-canonical pathway that does not require certain core components of the autophagy machinery, such as Atg5 or Atg7 (Kundu, et al. (2008) Blood 112:1493-1502; Honda, et al. (2014) Nat. Commun. 5:4004; Nashida, et al. (2009) Nature 461(7264):654-8). In addition, loss of the autophagy-activating Ulk1 gene in β-thalassemic mice reduces autophagic clearance of free α-globin in red blood cell precursors and exacerbates disease phenotypes (Lechauve, et al. (2019) Sci. Transl. Med. 11:eaav4881).

### SUMMARY OF THE INVENTION

The present invention provides methods for removing excess free α-globin and treating a thalassemia using an effective amount of a miR-144 and/or miR-451 antagomir. In certain embodiments, the thalassemia is β-thalassemia.

### BRIEF DESCRIPTION OF THE DRAWINGS

FIG. 1 shows red blood cell (RBC) counts in Hbb gene-disrupted β-thalassemic mouse model (Hbb^{Th3/+}) with (miR-144/451^{+/+} or without (miR-144/451^{-/-}) miR-144/451 expression.

FIG. 2 shows that reticulocyte counts in Hbb gene-disrupted β-thalassemic mouse model (Hbb^{Th3/+}) with (miR-144/451^{+/+}) or without (miR-144/451^{-/-}) miR-144/451 expression.

FIG. 3 shows RBC distribution width (RDW) in Hbb gene-disrupted β-thalassemic mouse model (Hbb^{Th3/+}) with (miR-144/451^{+/+}) or without (miR-144/451^{-/-}) miR-144/451 expression.

FIG. 4 shows the quantification of insoluble α-globin in Hbb gene-disrupted β-thalassemic mouse model (Hbb^{Th3/+}) with (miR-144/451^{+/+} or without (miR-144/451^{-/-}) miR-144/451 expression.

FIG. 5 shows that areas of electron-dense α-globin inclusions in reticulocytes quantified by automated image analysis of electron micrographs.

FIG. 6 shows red blood cell (RBC) counts in Hbb^{Th3/+} mice with (miR-144/451^{+/+}) or without (miR-144/451^{-/-}) miR-144/451 and with (Ulk1^{+/+} or without (Ulk1^{-/-}) Ulk1 expression.

FIG. 7 shows that reticulocyte counts in Hbb^{Th3/+} mice with (miR-144/451^{+/+}) or without (miR-144/451^{-/-}) miR-144/451 and with (Ulk1^{+/+}) or without (Ulk1^{-/-}) Ulk1 expression.

FIG. 8 shows the quantification of insoluble α-globin in Hbb^{Th3/+}miR-144/451^{-/-} mice with (Ulk1^{+/+}) or without (Ulk1^{-/-}) Ulk1 expression.

FIG. 9 shows red blood cell (RBC) counts in Hbb^{Th3/+}miR-144/451^{-/-} mice with (Cab39 CTRL) or without (Cab39 RNP) Cab39 expression.

FIG. 10 shows that reticulocyte counts in Hbb^{Th3/+}miR-144/451^{-/-} mice with (Cab39 CTRL) or without (Cab39 RNP) Cab39 expression.

FIG. 11 shows RDW in Hbb^{Th3/+}miR-144/451^{-/-} mice with (Cab39 CTRL) or without (Cab39 RNP) Cab39 expression.

### DETAILED DESCRIPTION OF THE INVENTION

The accumulation of free α-globin is a major determinant of the pathophysiology of β-thalassemia. Genetic ablation of ULK1 in β-thalassemia mice results in increased accumulation of free α-globin and exacerbation of the anemia phenotypes. In addition, mTORC1 has been shown to inhibit ULK1 and rapamycin, an mTORC1 inhibitor, alleviates β-thalassemia by derepressing ULK1. Contrary to the suggestion that suppression of miR-144 and/or miR-451 might be effective in reducing erythropoiesis (see, e.g., US 2014/0011859 A1), it has now been found that genetic disruption of the red blood cell expressed microRNA locus miR-144/451 improves the maturation of β-thalassemia erythroid precursors, enhances the survival of circulating red cells, results in an improvement of anemia and causes a dramatic reduction in the severity of β-thalassemia. Based upon the results presented herein, it is posited that miR451 derepresses its direct target mRNA MO25/CAB39, which in turn, activates the LKB1>AMPK>ULK1 pathway to stimulate autophagy of free α-globin (Fang, et al. (2018) Haematologica 103:406; Xu, et al. (2019) Blood 133(23):2518-2528; WO 2019/084402 A1). Accordingly, the present invention provides compositions and methods for treating a thalassemia disorder using one or more antagomirs that decrease the expression or activity of miR-144 and/or miR-451.

MiR-144 and miR-451 are part of a bicistronic cluster that is highly expressed during erythrocyte development. The miR-144/451 locus encodes two highly conserved miRNAs: miR-144-3p and miR-451a (unless otherwise indicated, miR-144 and miR-451 referred to herein are miR-144-3p and miR-451a, respectively). See Table 1. Although normally erythrocyte formation occurs throughout life in response to cytokine signaling, it has been shown that mice lacking miR-451 or lacking the miR-144/451 cluster display a mild reduction in hematocrit, a mild erythroid differentiation defect, and enhanced damage of red blood cells and their precursors during oxidative stress (Patrick, et al. (2010) Genes Dev. 24:1614-1619; Yu, et al. (2010) Genes Dev. 24:1620-1623; Rasmussen, et al. (2010) J. Exp. Med. 207:1351-1358).

TABLE 1

| | | SEQ |
|---|---|---|
| miRNA | miR sequence (5′−>3′) | ID NO: |
| Human pre- | UGGGGCCCUGGCUGGGAUAUCAUCAUAUACUG | 1 |
| mir-144 | UAAGUUUGCGAUGAGACACUACAGUAUAGAUG | |
| | AUGUACUAGUCCGGGCACCCCC | |
| Human pre- | CUUGGGAAUGGCAAGGAAACCGUUACCAUUAC | 2 |
| miR-451a | UGAGUUUAGUAAUGGUAAUGGUUCUCUUGCUA | |
| | UACCCAGA | |
| Mature | UACAGUAUAGAUGAUGUACU | 3 |
| Human mir- | | |
| 144 | | |
| Mature | AAACCGUUACCAUUACUGAGUU | 4 |
| Human miR- | | |
| 451a | | |
| Mouse pre- | GGCUGGGAUAUCAUCAUAUACUGUAAGUUUGU | 5 |
| mir-144 | GAUGAGACACUACAGUAUAGAUGAUGUACUAG | |
| | UC | |
| Mouse pre- | CUUGGGAAUGGCGAGGAAACCGUUACCAUUAC | 6 |
| miR-451a | UGAGUUUAGUAAUGGUAACGGUUCUCUUGCUG | |
| | CUCCCACA | |
| Mature | UACAGUAUAGAUGAUGUACU | 3 |
| Mouse mir- | | |
| 144 | | |
| Mature | AAACCGUUACCAUUACUGAGUU | 4 |
| Mouse miR- | | |
| 451a | | |

The term “thalassamia” or “thalassamia disorder” refers to a group of inherited autosomal recessive blood disorders that are common in various areas of the world including Mediterranean regions, India and Southeast Asia. In thalassemia, the genetic defect, which could be either a point mutation or deletion, results in a reduced rate of synthesis or no synthesis of one of the globin chains that make up hemoglobin. This can cause toxic buildup of the unaffected chain and also inhibit the production of normal hemoglobin, both causing anemia, the characteristic presenting symptom of the thalassemias. The two major forms of the disorder are alpha- and beta-thalassamia. “Beta-thalassamia” or “β-thalassemia” is a common inherited hemoglobinopathy characterized by impaired or absent β-globin gene production with consequent accumulation of unpaired α-subunits. The excess of unbound free α-globins precipitate in maturing erythroid cells and induces the production of reactive oxygen species (ROS) resulting in cellular oxidative stress damage and death of erythroid precursors, a process termed ineffective erythropoiesis. The presence of α-globin precipitates is also associated with a reduced RBC half-life and the clinical features of β-thalassemia, highlighting the importance of α-globin precipitates in the pathogenesis of the disease. By comparison “alpha-thalassemia” or “α-thalassemia” is a form of thalassemia involving the genes HBA1 and HBA2. Alpha-thalassemia is due to impaired production of 1, 2, 3 or 4 α-globin chains, leading to a relative excess of β-globin chains. The degree of impairment is based on which clinical phenotype is present (how many chains are affected). In certain embodiments, the subject treated in accordance with the methods described herein has β-thalassemia.

The subject treated in accordance with the methods described herein can be any mammal, including, but not limited to, humans, murines, simians, felines, canines, equines, bovines, mammalian farm animals, mammalian sport animals, and mammalian pets. Ideally, the subject is a human subject. In certain embodiments, the methods described herein are used to treat β-thalassemia in a subject, such as transfusion-dependent β-thalassemia (i.e., “Cooley's anemia”), non-transfusion-dependent β-thalassemia, β-thalassemia major, β-thalassemia intermedia, β-thalassemia minor, or β-thalassemia with associated hemoglobin (Hb) abnormalities (e.g., HbC/β-thalassemia, HbE/β-thalassemia, HbS/β-thalassemia). Thalassemia major, also referred to as transfusion-dependent β-thalassemia, is characterized by reduced Hb level (<7 g/dl), mean corpuscolar volume (MCV) >50<70 fl and mean corpuscolar Hb (MCH) >12<20 pg and requirements for regular red blood cell transfusions every 3-4 weeks. Thalassemia intermedia, also referred to as non-transfusion-dependent β-thalassemia, is characterized by Hb level between 7 and 10 g/dl, MCV between 50 and 80 fl and MCH between 16 and 24 pg and intermittent RBC transfusion requirements. Thalassemia minor is characterized by reduced MCV and MCH, with increased HbA2 (α₂δ₂) level.

“Treatment,” as used herein, refers to the application or administration of an antagomir (i.e., an antisense microRNA), or pharmaceutical composition containing the antagomir, to a subject, isolated tissue, isolated cells or cell line from a subject, where the subject has a thalassemia disorder, or a predisposition toward development of a thalassemia disorder, where the purpose is to cure, heal, alleviate, relieve, alter, remedy, ameliorate, improve, or affect the thalassemia disorder and/or any associated symptoms of the thalassemia disorder, or the predisposition toward the development of the thalassemia disorder. Ideally, treatment of a subject, tissue or cell will reduce anemia, ineffective erythropoiesis and/or transfusion burden in a subject with β-thalassemia.

An “effective amount” is an amount sufficient to effect beneficial or desired results. An effective amount can be administered in one or more administrations. An effective amount corresponds with the quantity required to provide a desired average local concentration of a particular biologic agent, in accordance with its known efficacy, for the intended period of therapy. A dose may be determined by those skilled in the art by conducting preliminary animal studies and generating a dose response curve, as is known in the art. Maximum concentration in the dose response curve would be determined by the solubility of the agent in the solution and by toxicity to the animal model, as known in the art.

In some embodiments, treatment of a subject with an antagomir of the invention removes excess free α-globin in erythroid cells by at least 20% as compared to free α-globin levels in the subject within 1, 2, 3, 4, 5, 6, 7, 8, 9, 10, 12, 13 or 14 weeks prior to the commencement of treatment of the subject. In certain embodiments, treatment removes excess free α-globin in erythroid cells of the subject by at least 50%. In certain embodiments, excess free α-globin is reduced in erythroid cells of the subject by at least 25%, 30%, 33%, 35%, 40%, 45%, 50%, 55%, 60%, 65%, 70%, 75%, 80%, 85%, 90%, 95%, 98%, or 100%. A reduction in free α-globin levels can be measured by its predicted downstream effects including increased RBC number, improved RBC morphology, enhanced survival of circulating red cells, reduced reticulocyte count, reduced ineffective erythropoiesis, reduced spleen size and reduced extramedullary erythropoiesis. In addition, the treatment would lead to a reduction in free α-globin levels determined by measuring α-globin levels before and after treatment using routine methods including, but not limited to cellulose acetate electrophoresis and DE-52 microchromatography, TRITON® acetic acid (TAU) urea gel electrophoresis and electron microscopy, high-performance liquid chromatography (HPLC), ELISA, western blot analysis, dot blot analysis and the like.

Subjects with thalassemia typically exhibit RBC morphologic changes (microcytosis, hypochromia, anisocytosis, poikilocytosis (spiculated tear-drop and elongated cells)), and nucleated RBC (i.e., red blood cells or erythroblasts). Accordingly, in certain embodiments, treatment of a subject according to the methods provided herein improves RBC morphology in the subject as compared to the RBC morphology in the subject within 1, 2, 3, 4, 5, 6, 7, 8, 9, 10, 11, 12, 13 or 14 weeks prior to the commencement of treatment of the subject according to the methods provided herein. Non-limiting determinants of improved RBC morphology include a 5% to 100% reduction in the ratio of number of abnormal RBCs in the subject to the total number of RBCs in the subject, a 5% to 100% reduction in the ratio of the number of RBCs with basophilic stippling in the subject to the total number of RBCs in the subject, a 5% to 100% reduction in the ratio of the number of poikilocytic RBCs in the subject to the total number of RBCs in the subject, a 5% to 100% reduction in the ratio of the number of schistocytes in the subject to the total number of RBCs in the subject, and a 5% to 100% reduction in the ratio of the number of irregularly contracted RBCs in the subject to the total number of RBCs in the subject within 1, 2, 3, or 4 weeks prior to the commencement of treatment of the subject.

For the purposes of this invention, an “antagomir” is a single-stranded, double stranded, partially double stranded or hairpin structured, that is optionally chemically modified, which consists of, consists essentially of or comprises at least 12 or more contiguous nucleotides substantially complementary to an endogenous miRNA and more particularly agents that include 12 or more contiguous nucleotides substantially complementary to a target sequence of an miRNA or pre-miRNA nucleotide sequence. The term “antagomir” can refer to chemical modifications to anti-miRNA molecules including locked nucleic acids (LNA), unlocked nucleic acids (UNA), 2′-O-methoxyethyl (2′-MOE) oligonucleotides, 2′-OMe oligonucleotides, 2′-Deoxy-2′-fluoro-nucleoside (2′-F) oligonucleotides, oligonucleotides with phosphodiester (PO) bonds, oligonucleotides with phosphorothioate (PS) linkages, phosphonoacetate (PACE) and thio-PACE linked oligonucleotides, phosphorodiamidate morpholino oligomers (PMO) and peptide nucleic acids (PNA). See Lima, et al. (2018) RNA Biol. 15(3):338-352; Petrescu, et al. (2019) J. Exp. Clin. Cancer Res. 38:231. As used herein partially double stranded refers to double stranded structures that contain less nucleotides than the complementary strand. In general, such partial double stranded agents will have less than 75% double stranded structure, preferably less than 50%, and more preferably less than 25%, 20% or 15% double stranded structure.

Preferably, an antagomir featured in the invention includes a nucleotide sequence sufficiently complementary to hybridize to a miRNA target sequence of about 12 to 25 nucleotides, preferably about 15 to 23 nucleotides. More preferably, the target sequence differs by no more than 1, 2, or 3 nucleotides from a sequence shown in Table 1, and in one embodiment, the antagomir is an agent shown in Table 2. In one embodiment, the antagomir includes a non-nucleotide moiety, e.g., a cholesterol moiety. The non-nucleotide moiety can be attached, e.g., to the 3′ or 5′ end of the oligonucleotide agent. In a preferred embodiment, a cholesterol moiety is attached to the 3′ end of the oligonucleotide agent.

In another aspect, the length of the antagomir can contribute to the biochemical function of the antagomir with respect to the ability to decrease expression levels of a desired miRNA. A miRNA-type antagomir can be, for example, from about 12 to 30 nucleotides in length, preferably about 15 to 28 nucleotides in length (e.g., 16, 17, 18, 19, 20, 21, 22, 23, 24, 25, 26, or 27 nucleotides in length). In some instances, antagomirs may require at least 19 nucleotides in length for optimal function.

The antagomir is further stabilized against nucleolytic degradation such as by the incorporation of a modification, e.g., a nucleotide modification. The antagomir includes a phosphorothioate at least the first, second, or third internucleotide linkage at the 5′ or 3′ end of the nucleotide sequence. In one embodiment, the antagomir includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In a particularly preferred embodiment, the antagomir includes at least one 2′-O-methyl-modified nucleotide, and in some embodiments, all of the nucleotides of the antagomir include a 2′-O-methyl modification. In yet another preferred embodiment, the antagomir includes six phosphorothioate backbone modifications; two phosphorothioates are located at the 5′-end and four at the 3′-end. In a preferred embodiment, the antagimor comprises 19 nucleotides and six phosphorothioate backbone modifications.

The antagomir is further modified so as to be attached to a ligand that is selected to improve stability, distribution or cellular uptake of the agent, e.g., cholesterol. In a preferred embodiment, the antagimor comprises 17 to 23 nucleotides, six phosphorothioate backbone modifications and a ligand to improve stability, distribution or cellular uptake of the antagomir. The oligonucleotide antagomir can further be in isolated form or can be part of a pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different miRNA.

An antagomir that is substantially complementary to a nucleotide sequence of a miRNA can be delivered to a cell or a human to inhibit or reduce the activity of an endogenous miRNA, such as when aberrant or undesired miRNA activity, or insufficient activity of a target mRNA that hybridizes to the endogenous miRNA, is linked to a disease or disorder. In one embodiment, an antagomir featured in the invention has a nucleotide sequence that is substantially complementary to miR-451 or miR-144 (see Table 1). In a preferred embodiment, the antagomir has a nucleotide sequence as provided in Table 2.

TABLE 2

| Antagomir Sequence (5′−>3′) | SEQ ID NO: |
|---|---|
| AACUCAGUAAUGGUAACGGUUU | 7 |
| UGGUAACGGUUU | 8 |
| AAUGGUAACGGUUU | 9 |
| GUAAUGGUAACGGUUU | 10 |
| AGUAAUGGUAACGGUU | 11 |
| GUAAUGGUAACGGUU | 12 |
| UAAUGGUAACGGUUU | 13 |
| UAAUGGUAACGGUU | 14 |
| AGUACAUCAUCUAUACUGUA | 15 |

Antagomirs of use in the method of this invention include those that eliminate, reduce or decrease the expression or activity of miR-144 or miR-451 thereby modulating ULK1 and/or ULK2. Ideally, the antagomirs of the present invention are formulated together with a pharmaceutically acceptable carrier and provided as a pharmaceutical composition. Pharmaceutical formulations comprising the one or more antagomirs of the invention may be prepared for storage by mixing one or more antagomirs having the desired degree of purity with optional physiologically acceptable carriers, excipients or stabilizers (Remington: The Science and Practice of Pharmacy 20^{th} edition (2000)), in the form of aqueous solutions, lyophilized or other dried formulations. Therefore, the invention further relates to a lyophilized or liquid formulation containing one or more antagomirs that modulate ULK1/2. As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, oral, topical, transdermal, intranasal, spinal or epidermal administration (e.g., by injection or infusion). Depending on the route of administration, the antagomir(s) may be coated in a material to protect the antagomir(s) from the action of acids and other natural conditions that may inactivate the antagomir(s).

A pharmaceutical composition of the invention also may include a pharmaceutically acceptable antioxidant. Examples of pharmaceutically acceptable antioxidants include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydro xyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of an injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization micro filtration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of antagomir(s) that can be combined with a carrier material to produce a single dosage form may vary depending upon antagomir(s), the subject being treated, and the particular mode of administration. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. In accordance with this invention, daily doses of may be in the range of about 0.01 mg to 100 mg, or about 0.1 to 50 mg. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

Alternatively, the antagomir(s) of the invention can be administered as a sustained release formulation in which case less frequent administration is required. Dosage and frequency vary depending on the half-life of the compounds in the patient.

Actual dosage levels of the antagomir(s) in the pharmaceutical compositions of the present invention may be varied so as to obtain an amount of the antagomir(s) which is effective to achieve the desired therapeutic response for a particular patient, composition, and mode of administration, without being toxic to the patient. The selected dosage level will depend upon a variety of pharmacokinetic factors including the activity of the particular antagomir(s) of the present invention employed, the route of administration, the time of administration, the rate of excretion of the particular antagomir(s) being employed, the duration of the treatment, other drugs, compounds and/or materials used in combination with the particular compositions employed, the age, sex, weight, condition, general health and prior medical history of the patient being treated, and like factors well known in the medical arts.

A pharmaceutical composition of the present invention can be administered by one or more routes of administration using one or more of a variety of methods known in the art. As will be appreciated by the skilled artisan, the route and/or mode of administration will vary depending upon the desired results. Routes of administration for the agents according to the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Alternatively, the antagomir(s) of the invention can be administered by a nonparenteral route, such as a topical, epidermal or mucosal route of administration, for example, intranasally, orally, vaginally, rectally, sublingually or topically.

The following non-limiting examples are provided to further illustrate the present invention.

### Example 1: Material and Methods

Mice. The breeding and analysis of Hbb^{Th3+/-}, Ulk1^{-/-}, and miR-144/45/^{-/-} mice were by routine methods. All mice were backcrossed onto the C57BL/6J background (The Jackson Laboratory, Bar Harbor, ME) for five to seven generations. Experiments were conducted with mice aged 6-24 weeks, with wild-type littermates used as controls.

Hematologic Analysis. Mice were analyzed at 2-6 months of age. Blood was collected by submandibular bleeding, anticoagulated with EDTA, and analyzed on a FORCYTE Veterinary Hematology Analyzer. Reticulocytes were quantified with thiazole orange (BD Biosciences) in accordance with the manufacturer's protocol, using an LSR/Fortess™ cell analyzer (BD Biosciences), and cell counts were analyzed using FlowJo 10.4.1 software (FlowJo, LLC).

Detection of Globin Precipitates in Erythroid Cells. Globin precipitates from erythrocytes were analyzed as described (Khandros, et al. (2012) Blood 119:5265-5275; Kong, et al. (2004) J. Clin. Invest. 114:1457-1466; Alter (1981) Prog. Clin. Biol. Res. 60:157-175; Yu, et al. (2007) J. Clin. Invest. 117:1856-1865; Sorensen, et al. (1990) Blood 75:1333-1336). Briefly, 20 μL of washed RBCs (normalized based on the hematocrit percentage) were lysed and centrifuged at 16,000×g at 4° C. for 30 minutes. The pellets were washed extensively in ice-cold 0.05× PBS. Membrane lipids were extracted with 56 mM sodium borate, pH 8.0, containing 0.1% surfactant sold under the tradename TWEEN®-20, at 4° C. Precipitated globins were dissolved in 8M urea, 10% acetic acid, 10% β-mercaptoethanol, and 0.04% pyronin, fractionated by TRITON™-acetic acid-urea (TAU) gel electrophoresis, and stained with Coomassie brilliant blue. Soluble hemoglobin fractions were analyzed and quantified as loading controls by using AlphaView SA 3.4.0 (ProteinSimple).

Electron Microscopy. Samples were fixed in 2.5% glutaraldehyde in 0.1M sodium cacodylate buffer, pH 7.4, and embedded in 2% low-gelling-temperature agarose. The samples were post-fixed in 2% osmium tetroxide in 0.1M cacodylate buffer with 0.3% potassium ferrocyanide for 1.5 hours, dehydrated through a graded series of ethanol/propylene oxide solutions, then infiltrated with and embedded in epoxy resin, which was polymerized at 70° C. overnight. Semi-thin (0.5 μm) sections were stained with toluidine blue for light microscope examination. Ultra-thin (80 nm) sections were cut and imaged using a FEI Tecnai 200 Kv FEG Transmission Electron Microscope with an ATM XR41 digital camera.

Pathology Analyses. Bright-field images of hematoxylin and eosin-stained tissues were acquired with a Nikon Eclipse Ni microscope. Morphologic phenotyping of all cohorts was carried out in a blinded manner by a board-certified veterinary pathologist. A semi-quantitative grading method was used to highlight visual differences in the erythropoiesis in each cohort. The bone marrow, spleen, and liver were first analyzed separately using the following grading scale: grade 0: within normal limits; grade 1: minimal; grade 2: mild; grade 3: moderate; grade 4: marked; grade 5: severe. A total grade for erythropoiesis (final grade) was calculated based on the sum of the grades for each of the three tissues. The final grading scale used to quantify the relative amount of erythropoiesis per mouse was as follows: grades 0-3 indicated that erythropoiesis was within what were considered to be the normal limits; grades 4-6 indicated a mild increase; grades 7-9 indicated a moderate increase; grades 10-12 indicated a marked increase; and grades 13-15 indicated a severe increase. No mouse received a grade consistent with a severe increase in erythropoiesis.

Statistics. Statistical analyses were performed using GraphPad Prism 9.2.0 software. For multiple comparisons, a one-way ANOVA with pairwise comparisons was used. A two-tailed Student's t-test was used for individual comparisons if they were normally distributed.

### Example 2: Genetic Ablation of miR-144/451 Locus in β-Thalassemia Mice

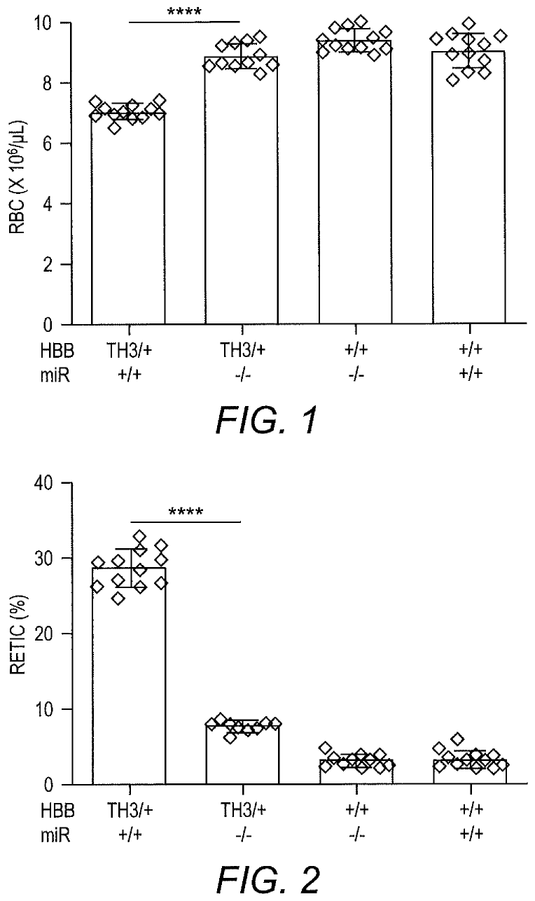

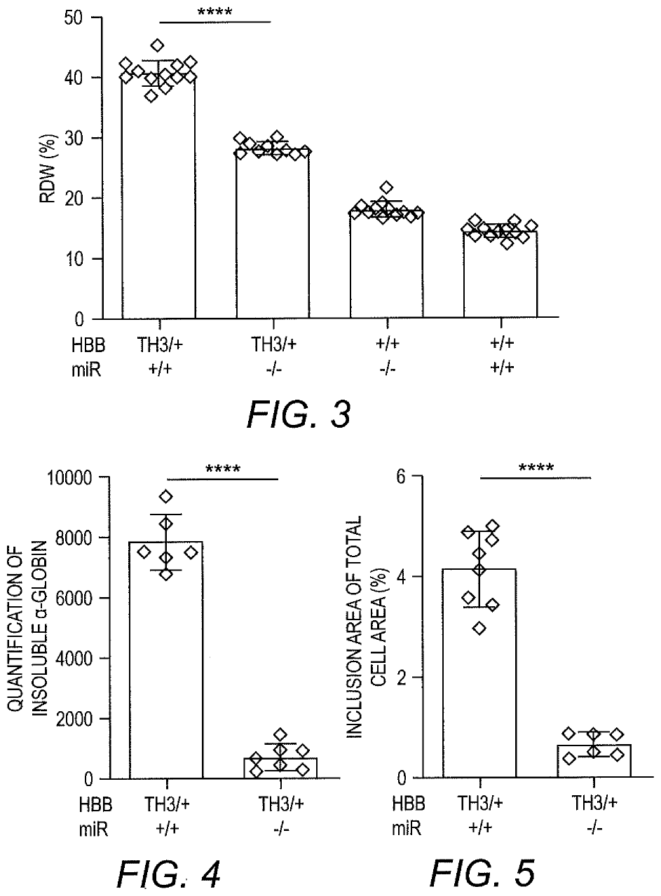

Hbb^{Th3/+} mice were crossed with the miR-144/451^{-/-} mice and evaluated for 6 months. The results of this analysis indicated that genetic disruption of the red blood cell expressed microRNA locus miR-144/451 improves the maturation of β-thalassemia erythroid precursors (FIG. 1) and reticulocyte count (FIG. 2) to maintain the same level of blood hemoglobin. In addition, disruption of the miR-144/451 locus enhances the survival of circulating red cells, reduces RDW (FIG. 3), decreases the amount of insoluble α-globin in RBCs (FIG. 4) and decreased the area of electron-dense material in Hbb^{Th3/+}miR-144/R451^{-/-} reticulocytes (FIG. 5). Notably, Hbb^{Th3/+}miR-144/451^{-/-} mice exhibited an improvement in spleen size and anemia, as well as a dramatic reduction in the severity of β-thalassemia.

### Example 3: miR-144/451 Activity is Dependent on ULK1

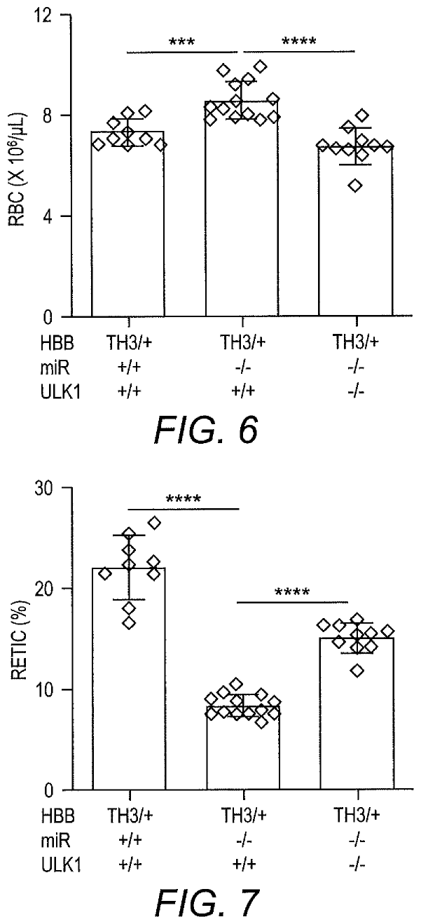

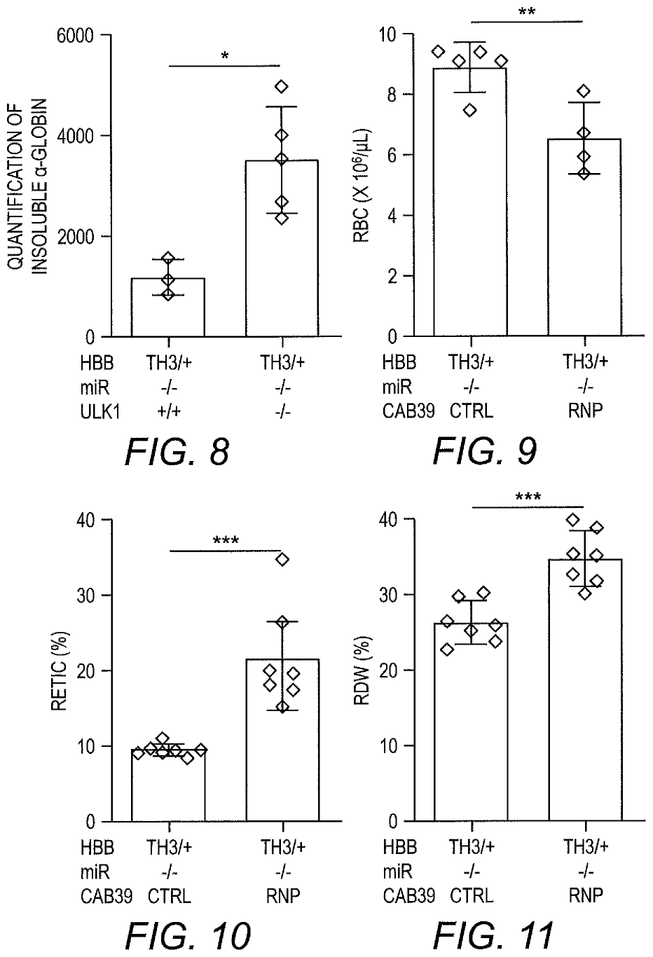

Hbb^{Th3/+}miR-144/451^{-/-} mice were crossed with the Ulk1^{-/-} mice and evaluated. The results of this analysis indicated that improvements in the maturation of β-thalassemia erythroid precursors (FIG. 6), reticulocyte count (FIG. 7) and decreases the amount of insoluble α-globin in RBCs (FIG. 8) observed in Hbb^{Th3/+}miR-144/451^{-/-} mice were dependent upon the expression of Ulk1.

### Example 4: miR-144/451 Activity is Dependent on CAB39

Hbb^{Th3/+} mice were crossed with the miR-144/451^{-/-} mice and miR-144/451^{-/-}Hbb^{Th3/+} hematopoietic stem cells were isolated by MACS microbeads purification. These cells were electroporated with ribonucleoprotein (RNP) complex composed of Cab39 guide RNAs and CAS9 proteins or electroporated with CAS9 alone (CTRL), then transplanted into lethally irradiated wild-type recipients. After 60 days, the animals were euthanized and analyzed. The results of this analysis indicated that improvements in the maturation of β-thalassemia erythroid precursors (FIG. 9), reticulocyte count (FIG. 10) and decreases RDW (FIG. 11) observed in Hbb^{Th3/+}miR-144/451^{-/-} mice were dependent upon the expression of Cab39. Accordingly, the data collectively indicate that loss of miR-144/451 derepresses the miR-451 direct target mRNA MO25/CAB39, which in turn, activates the LKB1>AMPK>ULK1 pathway to stimulate autophagy of free α-globin (Fang, et al. (2018) Haematologica 103:406; Xu, et al. (2019) Blood 133(23):2518-2528; WO 2019/084402 A1).

### Example 5: Antisense Oligonucleotide Treatment with Truncated Inhibitors of miR-451

A series of synthetic oligonucleotides targeting the mature miR-451 sequence are designed that range in length from 8 to 16 nucleotides to target the seed region of the microRNA and extend systematically toward the more 3′ end of the microRNA. These oligonucleotides may contain one or more bicyclic nucleosides (e.g., LNAs) and their sequences are listed in Table 3.

TABLE 3

| Oligo | Sequence (5′−>3′) | SEQ ID NO: |
|---|---|---|
| 8 nt oligo | AACGGUUU | 16 |
| 10 nt oligo | GUAACGGUUU | 17 |
| 12 nt oligo | UGGUAACGGUUU | 8 |
| 14 nt oligo | AAUGGUAACGGUUU | 9 |
| 16 nt oligo | GUAAUGGUAACGGUUU | 10 |

In addition to the five oligonucleotides listed above, anti-miR-451 oligonucleotides ranging from having a sequence that is complementary to the mature miR-451 sequence are also prepared (Table 4). All nucleosides in these anti-miR-451 oligonucleotides may be 2′-OMe modified and contain phosphorothioate internucleosides linking all bases.

TABLE 4

| Sequence (5′−>3′) | SEQ ID NO: |
|---|---|
| AGUAAUGGUAACGGUU | 18 |
| GUAAUGGUAACGGUU | 19 |
| UAAUGGUAACGGUUU | 20 |
| UAAUGGUAACGGUU | 21 |
| UAACGGUU | 22 |

### Example 6: CAB39 and LKB1 Activators

Based upon the results presented herein, miR451 derepresses its direct target mRNA MO25/CAB39, which in turn, activates the LKB1>AMPK>ULK1 pathway to stimulate autophagy of free α-globin. Therefore, compounds that activate or stimulate MO25/CAB39 (Calcium binding protein 39) or LKB1 (liver kinase B1) are also of use in the methods herein for removing excess free α-globin and treating a thalassemia.

Compounds that activate LKB1 can include a structure of Formula 1:

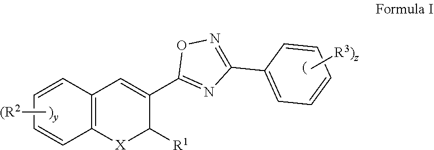

wherein R¹, R², and/or R³ are independently any substituent, X is O, N, or S; and y and z are independently 1, 2, 3, or 4 or 5. See U.S. Pat. No. 10,450,305 B2, incorporated herein by reference. An LKB1 activator may also include a catechin, capsaicin or flavanone (JP 2010215563 A), nootkatone (JP 2010280598 A), or rimonabant (Wu, et al. (2011) Mol. Pharmacol. 80(5):859-869).

What is claimed is:

1. A method for removing excess free α-globin in erythroid cells comprising contacting erythroid cells with an effective amount of (a) a miR-144 antagomir, (b) a miR-451 antagomir, or (c) a combination of (a) and (b), thereby removing excess free α-globin in the erythroid cells.

2. A method for treating a thalassemia comprising administering to a subject in need thereof an effective amount of an effective amount of (a) a miR-144 antagomir, (b) a miR-451 antagomir, or (c) a combination of (a) and (b), thereby treating the subject's thalassemia.

3. The method of claim 2, wherein the thalassemia is β-thalassemia.


SEQUENCE LISTING (22 sequences)

SEQ ID NO: 1 - RNA, 86 nt, Homo sapiens
uggggcccuggcugggauaucaucauauacuguaaguuugcgaugagacacuacaguauagaugauguacuaguccgggcaccccc

SEQ ID NO: 2 - RNA, 72 nt, Homo sapiens
cuugggaauggcaaggaaaccguuaccauuacugaguuuaguaaugguaaugguucucuugcuauacccaga

SEQ ID NO: 3 - RNA, 20 nt, Homo sapiens
uacaguauagaugauguacu

SEQ ID NO: 4 - RNA, 22 nt, Homo sapiens
aaaccguuaccauuacugaguu

SEQ ID NO: 5 - RNA, 66 nt, Mus musculus
ggcugggauaucaucauauacuguaaguuugugaugagacacuacaguauagaugauguacuaguc

SEQ ID NO: 6 - RNA, 72 nt, Mus musculus
cuugggaauggcgaggaaaccguuaccauuacugaguuuaguaaugguaacgguucucuugcugcucccaca

SEQ ID NO: 7 - RNA, 22 nt, Artificial Sequence
  Synthetic oligonucleotide
aacucaguaaugguaacgguuu

SEQ ID NO: 8 - RNA, 12 nt, Artificial Sequence
  Synthetic oligonucleotide
ugguaacgguuu

SEQ ID NO: 9 - RNA, 14 nt, Artificial Sequence
  Synthetic oligonucleotide
aaugguaacgguuu

SEQ ID NO: 10 - RNA, 16 nt, Artificial Sequence
  Synthetic oligonucleotide
guaaugguaacgguuu

SEQ ID NO: 11 - RNA, 16 nt, Artificial Sequence
  Synthetic oligonucleotide
aguaaugguaacgguu

SEQ ID NO: 12 - RNA, 15 nt, Artificial Sequence
  Synthetic oligonucleotide
guaaugguaacgguu

SEQ ID NO: 13 - RNA, 15 nt, Artificial Sequence
  Synthetic oligonucleotide
uaaugguaacgguuu

SEQ ID NO: 14 - RNA, 14 nt, Artificial Sequence
  Synthetic oligonucleotide
uaaugguaacgguu

SEQ ID NO: 15 - RNA, 20 nt, Artificial Sequence
  Synthetic oligonucleotide
aguacaucaucuauacugua

SEQ ID NO: 16 - RNA, 8 nt, Artificial Sequence
  Synthetic oligonucleotide
aacgguuu

SEQ ID NO: 17 - RNA, 10 nt, Artificial Sequence
  Synthetic oligonucleotide
guaacgguuu

SEQ ID NO: 18 - RNA, 16 nt, Artificial Sequence
  Synthetic oligonucleotide
aguaaugguaacgguu

SEQ ID NO: 19 - RNA, 15 nt, Artificial Sequence
  Synthetic oligonucleotide
guaaugguaacgguu

SEQ ID NO: 20 - RNA, 15 nt, Artificial Sequence
  Synthetic oligonucleotide
uaaugguaacgguuu

SEQ ID NO: 21 - RNA, 14 nt, Artificial Sequence
  Synthetic oligonucleotide
uaaugguaacgguu

SEQ ID NO: 22 - RNA, 8 nt, Artificial Sequence
  Synthetic oligonucleotide
uaacgguu